(19) United States
(12) Patent Application Publication
(10) Pub. No.: US 2005/0147975 A1
(43) Pub. Date: Jul. 7, 2005

(54) Methods and compositions for amplification of genomic DNA

(76) Inventors: Carol T. Schembri, San Mateo, CA (US); Leslie A. Leonard, Portola Way, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/746,871

(22) Filed: Dec. 24, 2003

(51) Int. Cl. C12Q 1/68; C12P 19/34

(52) U.S. Cl. 435/6; 435/91.2

(57) ABSTRACT

Methods for amplifying RNA from genomic DNA are provided. In the subject methods, a promoter-primer having a primer domain linked to an RNA polymerase promoter domain is first annealed to genomic DNA. The primer domain of the resultant annealed promoter-primer/genomic DNA complex is then extended to produce a double-stranded DNA molecule that has an RNA polymerase promoter domain. The resultant double-stranded DNA molecule is then transcribed into RNA product, e.g., labeled RNA product, using an RNA polymerase that is able to transcribe through the gap between the 5′ terminus of the promoter domain and the 3′ side of the genomic template. The subject methods find use a variety of different applications in which the preparation of amplified amounts of RNA from a genomic template is desired, where the amplification may be linear or geometric and may amplify the entire genome or only a select portion thereof. Also provided are kits for practicing the subject methods.

### TECHNICAL FIELD

The technical field of this invention is the enzymatic amplification of nucleic acids, particularly in the field of comparative genomic hybridization.

### BACKGROUND OF THE INVENTION

Many genomic and genetic studies are directed to the identification of differences in gene dosage or expression among cell populations for the study and detection of disease. For example, many malignancies involve the gain or loss of DNA sequences resulting in activation of oncogenes or inactivation of tumor suppressor genes. Identification of the genetic events leading to neoplastic transformation and subsequent progression can facilitate efforts to define the biological basis for disease, improve prognostication of therapeutic response, and permit earlier tumor detection. In addition, perinatal genetic problems frequently result from loss or gain of chromosome segments such as trisomy 21 or the micro deletion syndromes. Thus, methods of prenatal detection of such abnormalities can be helpful in early diagnosis of disease.

Comparative genomic hybridization (CGH) is one approach that has been employed to detect the presence and identify the location of amplified or deleted sequences. CGH reveals increases and decreases irrespective of genome rearrangement. In one implementation of CGH, genomic DNA is isolated from normal reference cells, as well as from test cells (e.g., tumor cells). The two nucleic acids are differentially labeled and then simultaneously hybridized in situ to metaphase chromosomes of a reference cell. Chromosomal regions in the test cells which are at increased or decreased copy number can be identified by detecting regions where the ratio of signal from the two DNAs is altered. For example, those regions that have been decreased in copy number in the test cells will show relatively lower signal from the test DNA than the reference compared to other regions of the genome. Regions that have been increased in copy number in the test cells will show relatively higher signal from the test DNA.

Currently, comparative genomic hybridization (CGH) protocols label the entire genome. While this approach may be necessary for broad screening assays, such approaches result in a highly complex mixture that may cause cross-hybridization and obscure the specific signals being sought.

Accordingly, there is interest in the development of improved methods of producing labeled nucleic acids from a genomic template, e.g., for use in CGH or other protocols.

### RELEVANT LITERATURE

United States patents of interest include: U.S. Pat. Nos. 6,132,997; 5,932,451; 5,716,785; 5,554,516; 5,545,522; 5,437,990; 5,130,238; and 5,514,545. See also Pollack et al., Nature Genetics (1999) 23: 41-46.

### SUMMARY OF THE INVENTION

Methods for amplifying RNA from genomic DNA are provided. In the subject methods, a promoter-primer having a primer domain linked to an RNA polymerase promoter domain is first annealed to genomic DNA. The primer domain of the resultant annealed promoter-primer/genomic DNA complex is then extended to produce a double-stranded DNA molecule that has an RNA polymerase promoter domain. The resultant double-stranded DNA molecule is then transcribed into RNA product, e.g., labeled RNA product, using an RNA polymerase that is able to transcribe through the gap between the 5′ terminus of the promoter domain and the 3′ side of the genomic template. The subject methods find use in a variety of different applications in which the preparation of amplified amounts of RNA from a genomic template is desired, where the amplification may be linear or geometric and may amplify the entire genome or only a select portion thereof. Also provided are kits for practicing the subject methods.

### BRIEF DESCRIPTION OF THE FIGURE

FIG. 1 provides a flow diagram of the general method of the subject invention.

### DEFINITIONS

The term “nucleic acid” as used herein means a polymer composed of nucleotides, e.g., deoxyribonucleotides or ribonucleotides, or compounds produced synthetically (e.g., peptide nucleic acids (PNA) as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to about 100 nucleotides and up to about 200 nucleotides in length.

The term “oligomer” is used herein to indicate a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other nucleic acids which are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “nucleic acid target element bound to a surface of a solid support” refers to an nucleic acid (poly or oligonucleotide) or mimetic thereof, e.g., peptide nucleic acids (PNA), that is immobilized on a surface of a solid substrate, where the substrate can have a variety of configurations, e.g., a sheet, bead, or other structure. In certain embodiments, the collections of nucleic acid target elements employed herein are present on a surface of the same planar support, e.g., in the form of an array.

The term “array” encompasses the term “microarray” and refers to an ordered array presented for binding to nucleic acids and the like.

An “array,” includes any one-dimensional, two-dimensional or substantially two-dimensional (as well as a three-dimensional) arrangement of addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain or via chemical linkers.

Any given substrate may carry one, two, four or more arrays disposed on a surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μ², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, light directed array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm. Alternatively, the substrate may be relatively reflective to reduce the absorption of the incident illuminating laser light and subsequent heating if the focused laser beam travels too slowly over a region. For example, the substrate may reflect at least 20%, or 50% (or even at least 70%, 90%, or 95%), of the illuminating light incident on the front as may be measured across the entire integrated spectrum of such illuminating light or alternatively at 532 nm or 633 nm

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, light directed array fabrication methods may be used. Inter-feature areas need not be present. Other fabrication methods of interest include physical compartmentalization in gel-based, e.g. acrylamide, materials.

An array is “addressable” when it has multiple regions of different moieties (e.g., different oligonucleotide sequences) such that a region (i.e., a “feature” or “spot” of the array) at a particular predetermined location (i.e., an “address”) on the array will detect a particular probe sequence. Array features are typically, but need not be, separated by intervening spaces. In the case of an array in the context of the present application, the “probe” will be referenced in certain embodiments as a moiety in a mobile phase (typically fluid), to be detected by “targets” which are bound to the substrate at the various regions.

A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide or substrate scanned in each pass of the lens, between the first feature of interest, and the last feature of interest, even if there exist intervening areas that lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic, gel, such as acrylamide, and other materials are also suitable.

The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

The substrate may be flexible (such as a flexible web). When the substrate is flexible, it may be of various lengths including at least 1 m, at least 2 m, or at least 5 m (or even at least 10 m).

The term “rigid” is used herein to refer to a structure, e.g., a bottom surface or a cover that does not readily bend without breakage, i.e., the structure is not flexible.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent conditions” refers to conditions under which a probe will hybridize preferentially to its target subsequence, and to a lesser extent to, or not at all to, other sequences. Put another way, the term “stringent hybridization conditions” as used herein refers to conditions that are compatible to produce duplexes on an array surface between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mnM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions set forth the conditions which determine whether a nucleic acid is specifically hybridizes to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen for detailed descriptions of equivalent hybridization and wash conditions and for reagents and buffers, e.g., SSC buffers and equivalent reagents and conditions.

Stringent hybridization conditions are hybridization conditions that are at least as stringent as the above representative conditions. Other stringent hybridization conditions are known in the art and may also be employed, as appropriate.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least non-contiguous system components, e.g., separate components, which may be in the same or in different rooms or different buildings, and where the different roomes or buildings may be at least one mile, ten miles, or at least one hundred miles apart. “Communicating” information references transmitting the data representing that information as electronicsignals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

The term “sample” as used herein relates to a material or mixture of materials, typically, although not necessarily, in fluid form, containing one or more components of interest.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### DESCRIPTION OF THE SPECIFIC EMBODIMENTS

Methods for amplifying RNA from genomic DNA are provided. In the subject methods, a promoter-primer having a primer domain linked to an RNA polymerase promoter domain is first annealed to genomic DNA. The primer domain of the resultant annealed promoter-primer/genomic DNA complex is then extended to produce a double-stranded DNA molecule that has an RNA polymerase promoter domain. The resultant double-stranded DNA molecule is then transcribed into RNA product, e.g., labeled RNA product, using an RNA polymerase that is able to transcribe through the gap between the 5′ terminus of the promoter domain and the 3′ side of the genomic template. The subject methods find use a variety of different applications in which the preparation of amplified amounts of RNA from a genomic template is desired, where the amplification may be linear or geometric and may amplify the entire genome or only a select portion thereof. Also provided are kits for practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All publications mentioned herein are incorporated herein by reference for the purpose of describing and disclosing the invention components that are described in the publications which might be used in connection with the presently described invention.

As summarized above, the present invention provides methods of preparing amplified amounts of RNA from genomic DNA, as well as kits for use in practicing the subject methods. In further describing the present invention, the subject methods are discussed first in greater detail, followed by a review of representative applications in which the subject methods find use, as well as a review of representative kits for use in practicing the subject methods.

Methods

The subject invention provides methods for amplifying genomic DNA into RNA. The subject invention may be used to label and amplify specific sequences from genomic DNA in order to reduce the complexity of the sample. The resultant product may be subsequently hybridized to a DNA microarray. Alternatively, the product may be employed to label and amplify all of the DNA, as described in greater detail below, where the latter may be advantageous when the available genomic sample is very small. Accordingly, the subject invention provides methods of producing amplified amounts of RNA from genomic DNA. As such, the subject invention provides methods of producing amplified amounts of RNA from an initial amount of genomic DNA. By amplified amounts is meant that for each amplified initial genomic DNA molecule (or domain or region thereof), multiple corresponding antisense RNAs are produced. By corresponding is meant that the amplified RNA, and specifically the primer derived portion thereof, shares a substantial amount of sequence identity with the sequence of one of the strands of the initial genomic DNA (i.e. the sense or antisense strand), where “substantial amount” in certain embodiments means at least about 95%, such as at least about 98% and including at least 99%, where sequence identity is determined using the BLAST algorithm, as described in Altschul et al. (1990), J. Mol. Biol. 215:403-410 (using the published default setting, i.e. parameters w=4, t=17). Generally, the number of corresponding RNA molecules produced for each initial genomic DNA molecule (or domain or region thereof) during the subject amplification methods will be at least about 10, such as at least about 50 and including at least about 100, where the number may be as great as 600 or greater, but in certain embodiments does not exceed about 1000.

In the first step of the subject methods, genomic DNA template is purified from cells and typically fragmented by methods known in the art. By genomic template is meant the nucleic acids that are used as template in the primer extension reactions, as described more in the following sections. In many embodiments, the genomic template is a population of genomic deoxyribonucleic acid molecules, where by population is meant a collection of molecules in which at least two constituent members have nucleotide sequences that differ from each other, e.g., by at least about 1 basepair, by at least about 5 basepairs, by at least about 10 basepairs, by at least about 50 base pairs, by at least about 100 base pairs, by at least about 1 kb, by at least about 10 kb etc.

The number of distinct sequences in a population of molecules making up a given genomic template is typically at least 2, usually at least 10 and more usually at least 50, where the number of distinct molecules may be 1000, 5000, 10000, 100000 or higher.

The genomic template may be prepared using any convenient protocol. In many embodiments, the genomic template is prepared by first obtaining a source of genomic DNA, e.g., a a nucleic acid containingfraction of a cell-lysate, where any convenient means for obtaining such a fraction may be employed and numerous protocols for doing so are well known in the art. The genomic template may be genomic DNA representing the entire genome from a particular organism, tissue or cell type or may comprise a portion of the genome, such as a single chromosome. Genomic template may be prepared from a subject, for example a plant or an animal that is suspected of being homozygous or heterozygous for a deletion or amplification of a genomic region. In many embodiments, the average size of the fragmented constituent molecules that make up the genomic template do not exceed about 10 kb in length, typically do not exceed about 8 kb in length and sometimes do not exceed about 5 kb in length, such that the average length of molecules in a given genomic template composition may range from about 1 kb to about 10 kb, usually from about 5 kb to about 8 kb in certain embodiments. The genomic template may be prepared from an initial chromosomal source by fragmenting the source into the genomic template having molecules of the desired size range, where fragmentation may be achieved using any convenient protocol, including but not limited to: mechanical protocols, e.g., sonication, shearing, etc., chemical protocols, e.g., enzyme digestion, etc.

Following preparation of the genomic template, as described above, the prepared genomic template is employed in the preparation of amplified amounts of RNA in a protocol in which at least one primer, and often a mixture of different primers, are employed, where the one or more primers are promoter-primers that include a primer domain and a promoter domain, where the primer domain is a domain that is designed or intended to hybridize to region of genomic DNA in the genomic template, as described above. As such, the promoter-primers employed in these embodiments include: (a) a primer domain or region for hybridization to a genomic sequence; and (b) an RNA polymerase promoter region or domain 5′ of the primer domain/region that is in an orientation capable of directing transcription of RNA from the double stranded region produced upon extension of the primer domain.

In certain embodiments, the primer domain of the promoter-primer(s) employed in the subject methods is a gene-specific or region-specific primer, such that gene-specific/region-specific promoter-primers that include both a gene/region-specific primer domain and an RNA polymerase-promoter domain are employed. The specific promoter-primers employed in the subject methods of this embodiment include: (a) gene/region specific primer region for hybridization to a target genomic sequence; and (b) an RNA polymerase-promoter region 5′ of the primer region that is in an orientation capable of directing transcription of RNA from the double stranded region produced upon extension of the primer domain.

The gene/region specific primer domain of the promoter-primers employed in the subject methods of this embodiment is one that specifically recognizes (i.e., hybridizes to under stringent conditions) a unique or distinct preknown or suspected sequence found in the genomic template, where the recognized sequence is one that may appear only a single time in the genome, or two or more times. In many embodiments, the primer domain is known to hybridize to a particular sequence known to be present in the genome, and therefore is distinct from a random primer which has a sequence that is not necessarily known to appear in the genome.

The primer domain of the gene-specific promoter-primers employed in the subject methods is one that is of sufficient length to specifically hybridize to a distinct nucleic acid member of the genomic sample, where the length of the gene specific primers in certain embodiments is at least about 8 nt, such as at about least about 20 nt and may be as long as about 25 nt or longer, but in certain embodiments does not exceed about 50 nt. The gene specific primer domains of the subject primers are sufficiently specific to hybridize to complementary genomic template sequence during the generation of labeled nucleic acids under conditions sufficient for primer extension, which conditions are known by those of skill in the art. The number of mismatches between the gene-specific primer-domain sequences and their complementary template sequences to which they hybridize during the annealing step of the subject methods will generally not exceed 20 number %, usually will not exceed 10 number % and more usually will not exceed 5 number %.

The primers herein are selected to be “substantially” complementary to each specific sequence to be amplified, i.e.; the primers should be sufficiently complementary to hybridize to their respective targets. Therefore, the primer sequence need not reflect the exact sequence of the target, and can, in fact be “degenerate.” Non-complementary bases or longer sequences can be interspersed into the primer, provided that the primer sequence has sufficient complementarity with the sequence of the target to be amplified to permit hybridization and extension.

In certain embodiments of the subject invention, a set of a representational number of specific promoter primers is employed in the annealing step. Generally, the sets of specific promoter-primers will comprise primers that correspond to at least 20, usually at least 50 and more usually at least 75 distinct genomic sequences, where any two genomic sequences are considered distinct if they comprise a stretch of at least 100 nt in their RNA coding regions in which the sequence similarity does not exceed 98%, as determined using the FASTA algorithm at default settings. In certain embodiments, the number of different specific promoter-primers in the set of promoter-primers may range from about 20 to about 10,000, usually from about 50 to about 2,000 and more usually from about 75 to about 1,500.

In certain embodiments, instead of having a primer domain that is gene/region specific, as described above, the primer domain is a random primer, such that the promoter-primers employed in the subject methods are molecules that include a random primer domain and a promoter domain. The primer domain of the promoter-primers of this embodiment are primers of random sequence. The primers employed in these embodiments may vary in length, and in many embodiments range in length from about 3 to about 25 nt, sometimes from about 5 to about 20 nt and sometimes from about 5 to about 10 nt. The total number of random primers of different sequence that is present in a given population of random primers employed in many embodiments of the subject invention may vary, and depends on the length of the primers in the set. As such, in the sets of random primers of a set of primer promoters which include all possible variations, the total number of primers n in the set of primers that is employed is 4^{Y}, where Y is the length of the 30 primers. Thus, where the primer set is made up of 3-mers, Y=3 and the total number n of random primers in the set is 4³ or 64. Likewise, where the primer set is made up of 8-mers, Y=8 and the total number n of random primers in the set is 4⁸ or 65,536. In yet other embodiments, the only a portion of the total number of possible random primers may be present in a set, as desired. Typically, an excess of random primers is employed, such that in a given primer set employed in the subject invention, multiple copies of each different random primer sequence is present, and the total number of primer molecules in the set far exceeds the total number of distinct primer sequences, where the total number may range from about 1.0×10¹⁰ to about 1.0×10²⁰, such as from about 1.0×10¹³ to about 1.0×10¹⁷, e.g., 3.7×10¹⁵.

As summarized above, the promoter-primers also include an RNA polymerase promoter domain. By RNA polymerase promoter domain is meant region or domain of DNA that includes an RNA polymerase promoter sequence. The promoter domain may be a single stranded or double-stranded, e.g., hairpin, domain, depending on the particular embodiment of the invention. A number of RNA polymerase promoters may be used for the hairpin promoter region. Suitable promoter regions will be capable of initiating transcription from an operationally linked DNA sequence in the presence of ribonucleotides and an RNA polymerase under suitable conditions. The promoter will be linked in an orientation to permit transcription of RNA. The promoter region will usually comprise between about 15 and 250 nucleotides, preferably between about 17 and 60 nucleotides, from a naturally occurring RNA polymerase promoter or a consensus promoter region, as described in Alberts et al. (1989) in Molecular Biology of the Cell, 2d Ed. (Garland Publishing, Inc.). Of interest are both prokaryotic promoters and eukaryotic promoters, as well as phage or virus promoters. As used herein, the term “operably linked” refers to a functional linkage between the affecting sequence (typically a promoter) and the controlled sequence (the mRNA binding site). The promoter regions that find use are regions where RNA polymerase binds tightly to the DNA and contain the start site and signal for RNA synthesis to begin. A wide variety of promoters are known and many are very well characterized. Representation promoter regions of interest include T7, T3 and SP6 as described in Chamberlin and Ryan, The Enzymes (ed. P. Boyer, Academic Press, New York) (1982) pp 87-108. The promoter region should be one that is recognized by a polymerase that can transcribe through the gap of the product molecules, described in greater detail below.

As indicated above, in certain embodiments, the RNA polymerase promoter domain of the primer-promoter is a hairpin RNA polymerase-promoter domain. By hairpin RNA polymerase-promoter domain is meant a nucleic acid sequence that includes regions of self-complementarity such that the sequence may assume a hairpin configuration, where when the domain assumes a hairpin configuration, the hairpin includes a double-stranded RNA polymerase-promoter.

In yet other embodiments, the polymerase promoter domain may be a linear domain which includes only one strand of the double-stranded promoter recognized by the RNA polymerase. In these embodiments, the method further includes addition of the complementary strand of the promoter (as described in greater detail below), so that a double stranded promoter is produced prior to the transcription step (described in greater detail below). A linker oligonucleotide between the promoter and the primer domains may be present, and if, present, will typically include between about 5 and about 20 bases, but may be smaller or larger as desired.

The promoter-primers described above and throughout this specification may be prepared using any suitable method, such as, for example, the known phosphotriester and phosphite triester methods, or automated embodiments thereof. In one such automated embodiment, dialkyl phosphoramidites are used as starting materials and may be synthesized as described by Beaucage et al. (1981), Tetrahedron Letters 22, 1859. One method for synthesizing oligonucleotides on a modified solid support is described in U.S. Pat. No. 4,458,066. It is also possible to use a primer that has been isolated from a biological source (such as a restriction endonuclease digest).

In the annealing step of the subject methods, the promoter-primer or primers are annealed to the genomic template. As such, the promoter-primer or primer(s) are contacted with the template in an aqueous medium to produce a reaction mixture which is maintained under conditions sufficient for the primer domains to hybridize to their complementary genomic sequences, if present, in the genomic template. Prior to the annealing step, the genomic template is typically present as double-stranded DNA molecules. Accordingly, during this annealing step, the template strands are typically disassociated and then allowed to anneal in the presence of the promoter-primer(s). In this annealing step, the promoter-primer(s) may be contacted with the template before or after the genomic template has been disassociated. The temperature of the reaction mixture is then reduced so that complementary strands in the reaction mixture re-associate, and gene-specific primer domains of the promoter-primers hybridize to their complementary genomic sequences present in the reaction mixture.

As such, in the annealing step of the subject methods, the genomic template is typically first subjected to strand disassociation conditions, e.g., subjected to a temperature ranging from about 80° C. to about 100° C., usually from about 90° C. to about 95° C. for a period of time, and the resultant disassociated template molecules are then contacted with the primer molecules under annealing conditions, where the temperature of the template and primer composition is reduced to an annealing temperature of from about 20° C. to about 80° C., usually from about 37° C. to about 65° C. In certain embodiments, a “snap-cooling” protocol is employed, where the temperature is reduced to the annealing temperature, or to about 4° C. or below in a period of from about 1 sec to about 30 sec, usually from about 5 sec to about 10 sec.

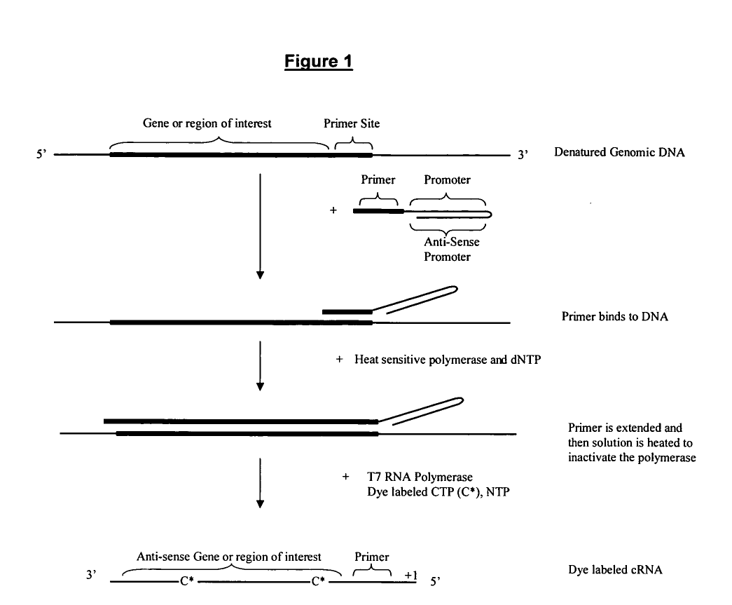

The above described annealing step results in the production of promoter-primer/genomic template complexes, where the complexes are characterized by having a primer domain hybridized to a complementary domain of a genomic template strand, and a RNA polymerase promoter domain. An additional feature of the complexes produced in this annealing step is that there is a gap between the 5′ terminus of the promoter primer and genomic template strand in the 3′ direction from the primer site, See e.g., FIG. 1. In the next step of the subject methods, the primer domain of any promoter-primer/genomic template complexes present in the reaction mixture is then extended to produce double-stranded DNA molecules that have an RNA polymerase promoter domain, which domain may be linear or duplex, e.g., hairpin, and may further be described as hanging, branched or dangling, as depicted in FIG. 1. The primer domain is extended in this step by maintaining the promoter-primer/genomic template complexes present in the reaction mixture under primer extension conditions for a sufficient period of time for primer extension to occur. As such, a DNA dependent DNA polymerase activity, deoxyribonucleotides (dATP, dGTP, dCTP, dTTP) and other additional reagents which include, but are not limited to: dNTPs; monovalent and divalent cations, e.g. KCl, MgCl₂; sulfhydryl reagents, e.g. dithiothreitol; and buffering agents, e.g. Tris-Cl, are provided in the reaction mixture and the reaction mixture is maintained at a suitable temperature for DNA dependent primer extension to occur.

The requisite DNA-dependent DNA polymerase activity may be provided by any convenient polymerase that exhibits such activity. In certain embodiments, the activity is provided by an enzyme that solely possesses the desired DNA dependent DNA polymerase activity, and does not possess any other activities, e.g., reverse transcriptase activity, etc. DNA dependent DNA polymerases of interest include, but are not limited to: a variety of DNA polymerases (such as those derived from E. coli, thermophilic bacteria, archaebacteria, phage, yeasts, Neurosporas, Drosophilas, primates and rodents. In yet other embodiments, the desired DNA-dependent DNA polymerase activity is provided by an enzyme that may include other activities as well, e.g., reverse transcriptase activity, RNAse H activity, and the like. For example, reverse transcriptases, such as those derived from Moloney murine leukemia virus (MMLV-RT) (as well as MMLV reverse transcriptase lacking RNaseH activity), avian myeloblastosis virus (AMV-RT), bovine leukemia virus (BLV-RT), Rous sarcoma virus (RSV) and human immunodeficiency virus (HIV-RT) can be employed to provide the DNA-dependent DNA polymerase activity. Suitable DNA polymerases possessing the desired activity may be isolated from an organism, obtained commercially or obtained from cells which express high levels of cloned genes encoding the polymerases by methods known to those of skill in the art, where the particular manner of obtaining the polymerase will be chosen based primarily on factors such as convenience, cost, availability, ability to be inactivated, and the like.

In this primer extension step, the resultant annealed primer/template hybrids are maintained in a reaction mixture that includes the above-discussed reagents at a sufficient temperature and for a sufficient period of time to produce the desired labeled probe nucleic acids. Typically, this incubation temperature ranges from about 20° C. to about 75° C., usually from about 37° C. to about 65° C. The incubation time typically ranges from about 5 min to about 18 hr, including from about 10 min to about 12 hr, such as from about 20 min to about 2 hr.

In certain embodiments, e.g., where the promoter-primer includes a single stranded promoter domain, the methods further include a step of contacting the promoter primer with a complement of the single stranded promoter domain in order to produce a double stranded pomoter domain for the in vitro transcription step, described in greater detail below. In these embodiments, oligonucleotides complementary to the singled stranded promoter domain of the promoter primers may be contacted with the promoter primers under annealing conditions at any convenient time prior to the in vitro transcription step, e.g., before primer extension, during primer extension, after primer extension, etc.

The above step results in the production of a double-stranded DNA molecule having a double stranded RNA polymerase promoter region, e.g., a hairpin promoter region, a non-hairpin promoter region produced by annealing of the promoter domain to a complementary oligonucleotide, etc., where the double stranded promoter region includes a double-stranded RNA polymerase-promoter domain. A feature of this product molecule is that there is a gap between the 5′ end of the double stranded promoter region and the 3′ direction of the genomic template strand of the structure, where this gap is typically no more than about 5 nt long, such as no more than about 3 nt long, including no more than about 2 or 1 nt long. As such, the product double-stranded DNA molecules include not only a sequence of nucleotide residues that comprises a DNA complement of the genomic template strand of the molecule, but also a terminal double-stranded promoter region. The double-stranded promoter region serves as a recognition site and transcription initiation site for RNA polymerase, which uses the synthesized primer extension strand (complementary to the genomic template strand) as a template for multiple rounds of RNA synthesis during the next stage of the subject methods.

The next step of the subject method is the preparation of RNA from the double-stranded DNA product of the first step. During this step, i.e., the in vitro transcription step, the double-stranded DNA molecules produced in the first step are transcribed by RNA polymerase to yield RNA product, which RNA product is complementary to the initial genomic template strand present in the amplified double-stranded molecules.

Depending on the particular protocol employed, the subject methods may or may not include a step in which the double-stranded DNA molecules produced as described above are physically separated from the polymerase activity employed in the dsDNA production step prior to the transcription step. For example, where the DNA-dependent DNA polymerase activity employed in the first step is provided by an enzyme having additional activities that are undesirable in the second transcription step, such a separation protocol may be employed to remove the polymerase. As such, in certain embodiments, the dsDNAs produced in the first step of the subject methods are separated from the polymerase activity employed in this first step prior to the second transcription step described in greater detail below. In these embodiments, any convenient separation protocol may be employed, including the phenol/chloroform extraction and ethanol precipitation (or dialysis), protocol as described in U.S. Pat. No. 5,554,516 and U.S. Pat. No. 5,716,785, the disclosures of which are herein incorporated by reference.

In yet other embodiments, removal of the undesirable activity of the polymerase employed in the first step may not include a separation step. Instead, the polymerase enzyme left over from the first step may be present during the transcription step, and where desirable may be rendered inactive, e.g., particularly if it includes a reverse transcriptase activity. Thus, the transcription step may be carried out in the presence of a polymerase activity that is unable to catalyze RNA-dependent DNA polymerase activity, at least for the duration of the transcription step. As a result, the RNA products of the transcription reaction cannot serve as substrates for additional rounds of amplification, and the amplification process cannot proceed exponentially, but instead proceeds linearly.

Where the DNA dependent DNA polymerase is provided by a reverse transcriptase, the reverse transcriptase present during the transcription step in these latter embodiments may be rendered inactive using any convenient protocol, including those described in U.S. Pat. No. 6,132,997; the disclosure of which is herein incorporated by reference. As described in this reference, the transcriptase may be irreversibly or reversibly rendered inactive. Where the transcriptase is reversibly rendered inactive, the transcriptase is physically or chemically altered so as to no longer able to catalyze RNA-dependent DNA polymerase activity. The transcriptase may be irreversibly inactivated by any convenient means. Thus, the reverse transcriptase may be heat inactivated, in which the reaction mixture is subjected to heating to a temperature sufficient to inactivate the reverse transcriptase prior to commencement of the transcription step. In these embodiments, the temperature of the reaction mixture and therefore the reverse transcriptase present therein is typically raised to 55° C. to 70° C. for 5 to 60 minutes, usually to about 65° C. for 15 to 20 minutes. Alternatively, reverse transcriptase may irreversibly inactivated by introducing a reagent into the reaction mixture that chemically alters the protein so that it no longer has RNA-dependent DNA polymerase activity. In yet other embodiments, the reverse transcriptase is reversibly inactivated. In these embodiments, the transcription may be carried out in the presence of an inhibitor of RNA-dependent DNA polymerase activity. Any convenient reverse transcriptase inhibitor may be employed which is capable of inhibiting RNA-dependent DNA polymerase activity a sufficient amount to provide for linear amplification. However, these inhibitors should not adversely affect RNA polymerase activity. Reverse transcriptase inhibitors of interest include ddNTPs, such as ddATP, ddCTP, ddGTP or ddTTP, or a combination thereof, the total concentration of the inhibitor typically ranges from about 50 μM to 200 μM.

Whether the methods include a step of removing the polymerase activity from the reaction mixture, e.g., by separation or inactivation, depends in part on whether linear or exponential amplification is desired. As such, in those embodiments where linear amplification is desired, the polymerase activity will generally be removed from the reaction mixture prior to transcription. In yet other embodiments where exponential amplificaiton is descired, the polymerase activity will not be removed.

For the transcription step, the presence of the double stranded RNA polymerase promoter region on the double-stranded DNA is exploited for the production of RNA. To synthesize the RNA, the double-stranded DNA is contacted with the appropriate RNA polymerase in the presence of the four ribonucleotides (i.e., UTP, ATP, GTP and CTP), under conditions sufficient for RNA transcription to occur, where the particular polymerase employed will be chosen based on the promoter region present in the double-stranded DNA, e.g. T7 RNA polymerase, T3 or SP6 RNA polymerases, E. coli RNA polymerase, and the like. Suitable conditions for RNA transcription using RNA polymerases are known in the art, see e.g. Milligan and Uhlenbeck (1989), Methods in Enzymol. 180, 51.

A key feature in the selection of the appropriate RNA polymerase is its ability to transverse a gap in the template strand. T7 RNA polymerase has been demonstrated to transverse gaps, nicks and branched junctions as described in Rong et al's “Template Stand Switching by T7 Ran Polymerase” The Journal of Biological Chemistry Vol 23 No. 17 pp 10253-10260 and in Zhou et al, “T7 RNA Polymerase Bypass of Large Gaps on the Template Strand Reveals a Critical Role of the Nontemplate Strand in Elongation” Cell, Vol 82, 577-525. As noted above, the DNA dependent RNA polymerase binds to the promoter site located on the double stranded promoter region. There is a gap between the 5′ terminus of the ds promoter and the genomic template strand. The RNA polymerase employed is one that has the ability to transcribe through this gap.

In certain embodiments, the RNA products of the above described transcription step are labeled. In these embodiments, the reagents employed in the subject transcription reactions typically include a labeling reagent, where the labeling reagent may be a directly or indirectly detectable label. A directly detectable label is one that can be directly detected without the use of additional reagents, while an indirectly detectable label is one that is detectable by employing one or more additional reagent, e.g., where the label is a member of a signal producing system made up of two or more components. In many embodiments, the label is a directly detectable label, such as a fluorescent label, where the labeling reagent employed in such embodiments is a fluorescently tagged nucleotide(s), e.g. fluorescently tagged CTP (such as Cy3-CTP, Cy5-CTP) etc. Fluorescent moieties which may be used to tag nucleotides for producing labeled probe nucleic acids include, but are not limited to: fluorescein, the cyanine dyes, such as Cy3, Cy5, Alexa 555, Bodipy 630/650, and the like. Other labels may also be employed as are known in the art.

The above protocol results in the production of an amplified population of RNA nucleic acids that are complementary to genomic templates primed by the specific primer(s) in the primer extension step. In certain embodiments, the product RNA nucleic acids are labeled, as described above. Where desired, the resultant RNA product nucleic acids may be separated from the remainder of the reaction mixture, where any convenient separation protocol may be employed.

In certain embodiments, the above protocol results in the production of a select population of RNA nucleic acids corresponding only to genes or regions of interest from an initial genomic template primed by the specific primers, and not to all of the template.

A representative protocol is shown in FIG. 1.

Utility

The resultant RNA nucleic acid populations produced by the above described methods find use in a variety of different applications. One broad type of application in which the product RNAs find use is nucleic acid analyte detection applications, where the subject methods may be employed to generate a labeled RNA nucleic acid analyte from an initial genomic template sample or source. Specific analyte detection applications of interest include hybridization assays in which the nucleic acids produced by the subject methods are hybridized to arrays of substrate bound nucleic acids. In these assays, a sample of labeled nucleic acids, e.g., labeled RNA or labeled deoxyribonucleic acids prepared from the RNA product, e.g., via reverse transcription etc., is first prepared according to the methods described above, where preparation may include labeling of the target nucleic acids with a label, e.g., a member of signal producing system. Following sample preparation, the sample is contacted with an array under hybridization conditions, whereby complexes are formed between solution phase nucleic acids that are complementary to substrate bound sequences attached to the array surface. The presence of hybridized complexes is then detected. An example of nucleic acid analyte detection applications of interest is the detection of polymorphisms of specific genes, where a tiling array of substrate bound nucleic acids (as described below) is employed with solution phase nucleic acids produced using one or more gene specific primers specific for the polymorphic region of interest.

A particular application of interest in which the subject RNA product nucleic acids have significant utility is in comparative genomic hybridization applications. In these applications, the above-amplified RNA production protocols are employed to produce collections or populations of labeled solution phase nucleic acids. The produced collections or populations of labeled solution phase nucleic acids are then contacted to a plurality of substrate bound elements under conditions such that nucleic acid hybridization to the substrate bound elements can occur. The solution phase collections can be contacted to the substrate bound elements either simultaneously or serially.

The substrate immobilized nucleic acids may range in size, and may be polynucleotides having lengths greater than about 200 nt, or oligonucleotides, where by oligonucleotide is meant a nucleic acid having a length ranging from about 10 to about 200 including from about 10 or about 20 to about 100 nt, where in many embodiments the substrate bound nucleic acids range in length from about 50 to about 90 nt or about 50 to about 80 nt, such as from about 50 to about 70 nt.

Substrate bound nucleic acids employed in such applications can be derived from virtually any source. Typically, they will be nucleic acid molecules having sequences derived from representative locations along a chromosome of interest, a chromosomal region of interest, an entire genome of interest, a cDNA library, and the like.

The choice of substrate bound nucleic acids to use may be influenced by prior knowledge or hypothesis of the association of a particular chromosome or chromosomal region with certain disease conditions. International Application WO 93/18186 provides a list of chromosomal abnormalities and associated diseases, which are described in the scientific literature. Alternatively, whole genome screening to identify new regions subject to frequent changes in copy number can be performed using the methods of the present invention. In these embodiments, substrate bound elements usually contain nucleic acids representative of locations distributed over the entire genome. In such embodiments, the resolution may vary, where in many embodiments of interest, the resolution is at least about 500 Kb, such as at least about 250 Kb, at least about 200 Kb, at least about 150 Kb, at least about 100 Kb, at least about 50 Kb, including at least about 25 Kb, at least about 10 Kb or higher. By resolution is meant the spacing on the genome between sequences found in the substrate bound elements. In some embodiments (e.g., using a large number of target elements of high complexity) all sequences in the genome can be present in the array. The spacing between different locations of the genome that are represented in the substrate bound elements of the collection of elements may also vary, and may be uniform, such that the spacing is substantially the same, if not the same, between sampled regions, or non-uniform, as desired.

In some embodiments, previously identified regions from a particular chromosomal region of interest are used as substrate bound nucleic acids. In certain embodiments, the array can include substrate bound elements which “tile” a particular region (which have been identified in a previous assay), by which is meant that the substrate bound nucleic acids correspond to region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled arrays tailored to the initially identified region to further assay the region at a higher resolution, e.g., in an iterative protocol.

Of interest are both coding and non-coding genomic regions, where by coding region is meant a region of one or more exons that is transcribed into an mRNA product and from there translated into a protein product, while by non-coding region is meant any sequences outside of the exon regions, where such regions may include regulatory sequences, e.g., promoters, enhancers, introns, etc. In certain embodiments, one can have at least some of the substrate bound elements directed to non-coding regions and others directed to coding regions. In certain embodiments, one can have all of the substrate bound elements directed to non-coding sequences. In certain embodiments, one can have all of the substrate bound elements directed to coding sequences.

In certain embodiments, the only substrate bound elements present on the array are ones that correspond to the specific primers employed in the probe generation step, described above, such that the array only includes substrate bound elements that hybridize to solution phase nucleic acids produced by the gene specific primers employed in the solution phase nucleic acid generation step.

The substrate bound elements employed in the subject methods are immobilized on a solid support. Many methods for immobilizing nucleic acids on a variety of solid support surfaces are known in the art. For instance, the solid support may be a membrane, glass, plastic, or a bead. The desired component may be covalently bound or non-covalently attached through nonspecific binding, adsorption, physisorption or chemisorption. The immobilization of nucleic acids on solid support surfaces is discussed more fully below.

A wide variety of organic and inorganic polymers, as well as other materials, both natural and synthetic, may be employed as the material for the substrate, or at least a surface thereof, e.g., a solid support surface. Illustrative materials of interest include nitrocellulose, nylon, glass, fused silica, diazotized membranes (paper or nylon), silicones, cellulose, and cellulose acetate. In addition, plastics such as polyethylene, polypropylene, polystyrene, and the like can be used. Other materials which may be employed include paper, ceramics, metals, metalloids, semiconductive materials, cermets or the like. In addition substances that form gels can be used. Such materials include proteins (e.g., gelatins), lipopolysaccharides, silicates, agarose and polyacrylamides. Where the substrate is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, enhance signal detection or the like.

If covalent bonding between a compound and the surface is desired, the surface will usually include appropriate functionalities to provide for the covalent attachment. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces are well known and is amply illustrated in the literature. For example, methods for immobilizing nucleic acids by introduction of various functional groups to the molecules is known (see, e.g., Bischoff et al., Anal. Biochem. 164:336-344 (1987); Kremsky et al., Nuc. Acids Res. 15:2891-2910 (1987)). Modified nucleotides can be placed on the target using PCR primers containing the modified nucleotide, or by enzymatic end labeling with modified nucleotides, or by non-enzymatic synthetic methods

Use of membrane supports (e.g., nitrocellulose, nylon, polypropylene) for the nucleic acid arrays of the invention is advantageous in certain embodiments because of well-developed technology employing manual and robotic methods of arraying targets at relatively high element densities (e.g., up to 30-40/cm²). In addition, such membranes are generally available and protocols and equipment for hybridization to membranes is well known. Many membrane materials, however, have considerable fluorescence emission, where fluorescent labels are used to detect hybridization.

To optimize a given assay format one of skill can determine sensitivity of fluorescence detection for different combinations of membrane type, fluorochrome, excitation and emission bands, spot size and the like. In addition, low fluorescence background membranes have been described (see, e.g., Chu et al., Electrophoresis 13:105-114 (1992)).

The sensitivity for detection of spots of various diameters on the candidate membranes can be readily determined by, for example, spotting a dilution series of fluorescently end labeled DNA fragments. These spots are then imaged using conventional fluorescence microscopy. The sensitivity, linearity, and dynamic range achievable from the various combinations of fluorochrome and membranes can thus be determined. Serial dilutions of pairs of fluorochrome in known relative proportions can also be analyzed to determine the accuracy with which fluorescence ratio measurements reflect actual fluorochrome ratios over the dynamic range permitted by the detectors and membrane fluorescence.

Arrays on substrates with much lower fluorescence than membranes, such as glass, quartz, or small beads, can achieve much better sensitivity. For example, elements of various sizes, ranging from the about 1 mm diameter down to about 1 μm can be used with these materials. Small array members containing small amounts of concentrated target DNA are conveniently used for high complexity comparative hybridizations since the total amount of probe available for binding to each element will be limited. Thus it may be advantageous in certain embodiments to have small array members that contain a small amount of concentrated target DNA so that the signal that is obtained is highly localized and bright. Such small array members are typically used in arrays with densities greater than 10⁴/cm². Relatively simple approaches capable of quantitative fluorescent imaging of 1 cm² areas have been described that permit acquisition of data from a large number of members in a single image (see, e.g., Wittrup et. al. Cytometry 16:206-213 (1994)).

Covalent attachment of the nucleic acids to glass or synthetic fused silica can be accomplished according to a number of known techniques. Such substrates provide a very low fluorescence substrate, and a highly efficient hybridization environment.

There are many possible approaches to coupling nucleic acids to glass that employ commercially available reagents. For instance, materials for preparation of silanized glass with a number of functional groups are commercially available or can be prepared using standard techniques. Alternatively, quartz cover slips, which have at least 10-fold lower auto fluorescence than glass, can be silanized. In certain embodiments of interest, silanization of the surface is accomplished using the protocols described in U.S. Pat. No. 6,444,268, the disclosure of which is herein incorporated by reference, where the resultant surfaces have low surface energy that results from the use of a mixture of passive and functionalized silanization moieties to modify the glass surface, i.e., they have low surface energy silanized surfaces. Additional linking protocols of interest include, but are not limited to: polylysine as well as those disclosed in U.S. Pat. No. 6,319,674, the disclosure of which is herein incorporated by reference. The targets can also be immobilized on commercially available coated beads or other surfaces. For instance, biotin end-labeled nucleic acids can be bound to commercially available avidin-coated beads. Streptavidin or anti-digoxigenin antibody can also be attached to silanized glass slides by protein-mediated coupling using e.g., protein A following standard protocols (see, e.g., Smith et al. Science, 258:1122-1126 (1992)). Biotin or digoxigenin end-labeled nucleic acids can be prepared according to standard techniques. Hybridization to nucleic acids attached to beads is accomplished by suspending them in the hybridization six, and then depositing them on the glass substrate for analysis after washing. Alternatively, paramagnetic particles, such as ferric oxide particles, with or without avidin coating, can be used.

In the subject methods (as summarized above), the copy number of particular nucleic acid sequences in two probe collections are compared by hybridizing the solution phase nucleic acids to one or more target nucleic acid arrays, as described above. The hybridization signal intensity, and the ratio of intensities, produced by the solution phase nucleic acids hybridized to each of the substrate bound elements is determined. Since signal intensities on a substrate bound element can be influenced by factors other than the copy number of a solution phase nucleic acid, for certain embodiments an analysis is conducted where two labeled populations are present with distinct labels. Thus comparison of the signal intensities for a specific substrate bound element permits a direct comparison of copy number for a given sequence. Different substrate bound elements will reflect the copy numbers for different sequences in the solution phase populations The comparison can reveal situations where each sample includes a certain number of copies of a sequence of interest, but the numbers of copies in each sample are different. The comparison can also reveal situations where one sample is devoid of any copies of the sequence of interest, and the other sample includes one or more copies of the sequence of interest. The comparison may also reveal polymorphisms in one region of a sample which do not appear in a second or control sample.

Standard hybridization techniques (using high stringency hybridization conditions) are used to probe a nucleic acid array. Suitable methods are described in references describing CGH techniques (Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For descriptions of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985). See also U.S. Pat. Nos: 6,335,167; 6,197,501; 5,830,645; and 5,665,549; the disclosures of which are herein incorporate by reference.

Generally, nucleic acid hybridizations comprise the following major steps: (1) immobilization of substrate bound nucleic acids; (2) pre-hybridization treatment to increase accessibility of target DNA, and to reduce nonspecific binding; (3) hybridization of the mixture of nucleic acids to the nucleic acid on the solid surface, typically under high stringency conditions; (4) post-hybridization washes to remove nucleic acid fragments not bound in the hybridization and (5) detection of the hybridized nucleic acid fragments. The reagents used in each of these steps and their conditions for use vary depending on the particular application.

As indicated above, hybridization is carried out under suitable hybridization conditions, which may vary in stringency as desired, where suitable hybridization conditions are described above.

The above hybridization step may include agitation of the immobilized nucleic acids and the sample of solution phase nucleic acids, where the agitation may be accomplished using any convenient protocol, e.g., shaking, rotating, spinning, and the like.

Following hybridization, the surface of immobilized nucleic acids is typically washed to remove unbound or non-specifically bound solution phase nucleic acids. Washing may be performed using any convenient washing protocol, where the washing conditions are typically stringent, as described above.

Following hybridization and washing, as described above, the hybridization of the labeled nucleic acids to the substrate bound nucleic acids is then detected using standard techniques so that the surface of immobilized nucleic acids, e.g., array, is read. Reading of the resultant hybridized array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable devices and methods are described in U.S. patent applications: Ser. No. 09/846,125 “Reading Multi-Featured Arrays” by Dorsel et al.; and U.S. Pat. No. 6,406,849, which references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of the probe nucleic acids, and are suitable for some embodiments.

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

The above-described methods find use in any application in which one wishes to compare the copy number of nucleic acid sequences found in two or more populations. One type of representative application in which the subject methods find use is the quantitative comparison of copy number of one nucleic acid sequence in a first collection of nucleic acid molecules relative to the copy number of the same sequence in a second collection.

As such, the present invention may be used in methods of comparing abnormal nucleic acid copy number and mapping of chromosomal abnormalities associated with disease. In many embodiments, the subject methods are employed in applications that use target nucleic acids immobilized on a solid support, to which differentially labeled probe nucleic acids produced as described above are hybridized. Analysis of processed results of the described hybridization experiments provides information about the relative copy number of nucleic acid domains, e.g. genes, in genomes.

Such applications compare the copy numbers of sequences capable of binding to the target elements. Variations in copy number detectable by the methods of the invention may arise in different ways. For example, copy number may be altered as a result of amplification or deletion of a chromosomal region, e.g., as may commonly occur in cancer. Representative applications in which the subject methods find use are further described in U.S. Pat. Nos. 6,335,167; 6,197,501; 5,830,645; and 5,665,549; the disclosures of which are herein incorporated by reference.

The subject methods find particular use in high resolution CGH applications where initially small sample volumes are to be analyzed, such as the small sample volumes described above. Small samples may be derived after purification of subpopulations of cells of interest from a starting tissue sample. For example, single and multi-parameter flow cytometry can identify small numbers of abnormal cells in a background of large numbers of normal cells in a biopsy or mixed cell population. Another technique that may be used to produce small samples of purified cells is laser capture microdissection. A particular advantage of the subject methods over labeling and hybridizing the entire genome to an array is a significant reduction in the complexity of the sample, which may reduce the level of cross-hybridization and non-specific hybridization on the array, thereby lowering the noise, which increases the signal-to-noise making the subsequent analysis easier.

Kits

Also provided are kits for use in the subject invention, where such kits may include containers, each with one or more of the various reagents (typically in concentrated form) utilized in the methods, including, for example, buffers, the appropriate nucleotide triphosphates (e.g. dATP, dCTP, dGTP, dTTP, ATP, CTP, GTP and UTP), DNA dependent DNA polymerase, RNA polymerase, and the promoter-primers of the present invention. Also present in the kits may be prefabricated arrays, where the features of the arrays may be limited to ones that correspond to the gene specific primers present in the kits.

The kits may further include instructions for using the kit components in the subject methods. The instructions may be present in the kits as a package insert, in the labeling of the container of the kit or components thereof (i.e., associated with the packaging or sub-packaging) etc., and may be printed on a substrate, such as paper or plastic. In other embodiments, the instructions are present as an electronic storage data file present on a suitable computer readable storage medium, e.g., CD-ROM, diskette, etc.

The following examples are offered by way of illustration and not by way of limitation.

### EXPERIMENTAL

### I. Preparation of Genomic Template

A genomic template is prepared using a Qiagen Blood and Cell Culture DNA Maxi Kit as described in Pollack et al., Nature Genetics (1999) 23: 41-46.

### II. Annealing of Specific Probes with Dangling T7 Promoter Site

1. Add 2 μg DNA of the sample to be labeled into an eppendorf tube.

2. Add water to bring total volume to 20 μl.

3. Add 4 μl of the specific T7 promoter primers for the application.

4. Boil 5 minutes, then place on ice.

### III. Create dsDNA in Those Sites

1. Mix 8 μl 5×first strand buffer (250 mM Tris-HCL, pH 83, 15 mM MgCl₂, 375 nM KCl) 4 μl 0.1 DTT, 2 μl 10 mM dNTP mix and 2 μl MMLV-RT.

2. Add 16 μl of mixture to the sample tube. Incubate DNA synthesis reaction at 40° C. for 20 minutes-2 hours.

3. Incubate the mixture at 65° C. for 15 minutes to inactivate the enzyme.

### IV. Create Labeled RNA

1. Prepare transcription mix:
nuclease free water—41.6 μl
5×Transcription buffer—32 μl (0.2M Tris-HCl, pH 7.5, 50 mM NaCl, 30 mM MgCl₂, 10 mM spermidine)
100 mM DTT—12 μl
NTPs (25 mM A, G, U, 7.5 mM CTP)—16 μl
Cy3-CTP or Cy5-CTP (7.0 mM)—8 μl
200 mM MgCl2—6.6 μl
RNA Guard—1 μl
Inorganic pyrphosphatase (200 U/ml)—1.2 μl
T7 RNA polymerase (2500 U/μl)—1.6 μl

2. Aliquot 120 μl of mixture to the sample tube. Incubate transcription reaction for 60 minutes at 40° C.

### V. Precipitate the RNA

1. Add 160 μl 4M LiCl and place the tube in the −20° C. freezer for one hour to overnight.

2. Spin the LiCl precipitates at 4° C. in the microcentrifuge.

3. Rinse each sample pellet in 70% ethanol. Dry briefly at room temperature.

4. Resuspend each sample pellet in 100 μl nuclease free water.

5. Quantify sample using 10D₂₆₀=40 μg/ml RNA.

### VI. Hybridization Reaction

Hybridization was carried out according to the protocol described in the Agilent in-situ microarray hybridization protocol user's guide; available under publication number G4140-90030 from Agilent Technologies (Palo Alto, Calif.).

The above described invention provides a way of generating amplified amounts of RNA nucleic acids from a genomic template, where the inventive methods and compositions find use in a variety of applications, including CGH applications. With respect to CGH applications, benefits include the ability to achieve higher sensitivity with reduced initial sample size. As such, the subject methods represent a significant contribution to the art.

All publications and patent application cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for producing amplified amounts of RNA from genomic DNA, said method comprising:
(a) contacting a genomic DNA source with at least one promoter-primer under annealing conditions to produce a primed genomic DNA sample, wherein said promoter-primer comprises a primer domain linked to a RNA polymerase-promoter domain;
(b) subjecting said primed genomic DNA sample to primer extension reaction conditions to extend a primer domain of any resultant promoter-primer/genomic DNA complexes to produce double-stranded DNA molecules having a RNA polymerase-promoter domain; and
(c) transcribing RNA from any resultant double-stranded DNA molecules having a RNA polymerase-promoter to produce amplified amounts of RNA from genomic DNA.

2. The method according claim 1, wherein said promoter-primer is a gene-specific promoter-primer.

3. The method according to claim 1, wherien said promoter-primer is a random promoter-primer.

4. The method according to claim 1, wherein said promoter-primer comprises a hairpin promoter domain.

5. The method according to claim 1, wherein said promoter-primer comprises a double-stranded promoter domain.

6. The method according to claim 1, wherein said method is a method of producing linearly amplified amounts of RNA.

7. The method according to claim 1, wherein said method is a method of producing exponentially amplified amounts of RNA.

8. The method according to claim 1, wherein said genomic DNA sample is fragmented prior to contact with said at least one gene-specific promoter-primer.

9. The method according to claim 1, wherein said genomic DNA sample is contacted with a set of different promoter-primers, wherein each constituent member of said set has a different primer domain.

10. The method according to claim 1, wherein said RNA polymerase promoter domain is a T7 promoter domain.

11. A method of detecting the presence of a nucleic acid analyte in a sample comprising:
(a) contacting said sample with a nucleic acid array, wherein said sample is a sample of amplified amounts of RNA produced from genomic DNA according to the method of claim 1;
(b) detecting any binding complexes on the surface of said array to obtain binding complex data; and
(c) determining the presence of said nucleic acid analyte in said sample using said binding complex data.

12. The method according to claim 11, wherein said method further comprises a data transmission step in which a result from a reading of the array is transmitted from a first location to a second location.

13. A method according to claim 12, wherein said second location is a remote location.

14. A method comprising receiving data representing a result obtained by the method of claim 11.

15. A method for comparing the copy number of at least one nucleic acid sequence in at least two genomic sources, said method comprising:
(a) producing amplified amounts of solution phase nucleic acids from a first genomic template from a first genomic source and a second genomic template from a second genomic source according to the method of claim 1 to produce a first and a second collection of solution phase nucleic acids;
(b) contacting said first and second collections of nucleic acids with one or more pluralities of nucleic acid elements bound to a surface of a solid support, each element comprising a nucleic acid; and
(c) evaluating the binding of the first and second collections of solution phase nucleic acid molecules to the same support bound nucleic acid to compare the copy number of at least one nucleic acid sequence in said at least two genomic sources.

16. The method according to claim 15, wherein the solid support is a planar substrate.

17. The method according to claim 15, wherein said method is a comparative genomic hybridization method.

18. The method according to claim 15, wherein said method further comprises a data transmission step in which a result from said evaluating is transmitted from a first location to a second location.

19. The method according to claim 18, wherein said second location is a remote location.

20. A method comprising receiving data representing a result obtained by the method of claim 15.

21. A kit for use in amplifying RNA from genomic DNA, said kit comprising:
a promoter-primer comprising a primer domain linked to an RNA polymerase-promoter domain; and instructions for practicing the method according to claim 1.

22. The kit according to claim 21, wherein said kit further comprises at least one DNA polymerase.

23. The kit according to claim 21, wherein said kit further comprises an RNA polymerase.

24. A gene-specific promoter-primer comprising a primer domain linked to a hairpin RNA polymerase promoter domain.
